(19) United States
(12) Patent Application Publication
(10) Pub. No.: US 2005/0074476 A1
(43) Pub. Date: Apr. 7, 2005

(54) Cartilidge and bone induction by artificially perforated organic bone matrix augmented by undifferentiated cells suspended in bone gel

(76) Inventors: El Gendler, Santa Monica, CA (US); Eli Gendler, Santa Monica, CA (US); Simon Gendler, Los Angeles, CA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/679,234

(22) Filed: Oct. 3, 2003

(51) Int. Cl. A61F 2/00; C12N 5/02

(52) U.S. Cl. 424/422; 424/423; 435/395

(57) ABSTRACT

A novel method for cartilage and bone induction is introduced whereby an artificially perforated bone organic bone matrix augmented by live human undifferentiated cells suspended in a gel is used as a surgical implant.

### FIELD OF THE INVENTION

This invention pertains to organic bone matrices augmented with undifferentiated cells suspended in bone gel and useful in surgical implantation for the purpose of causing cartilage and bone induction, as well as to processes for making same, and methods of using same.

### BACKGROUND OF THE INVENTION

As is known, bones and teeth are composed of a matrix of organic material consisting of collagenous fibrils and a binding substance of mucopolysaccharides as well as of the inorganic component, namely calcium phosphate in the form of hydroxyapatite. The organic matrix is formed by filiform molecules arranged parallel to each other. Furthermore, the tissue is transversed by numerous microscopic capillaries which are oriented in various directions to said filiform molecules.

It is known that if the inorganic component is partially or completely removed form the bone or tooth, the remaining organic bone material, called organic bone matrix, can be transplanted to other living animal or human bodies without substantial deleterious effects. Consequently, bone matrix is used in modern medical procedures for its ability to induce formation of cartilage and bone after its implantation into a body site (this phenomena is known as “osteoinduction”).

Research has been conducted on undifferentiated cells, or stem cells, for their noted ability to grow specialized cells or tissue, which could be used to treat injuries or disease. In general, undifferentiated cells, or stem cells, are cells that can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

Stem cells are used as a source for alternative treatments of disease or injury to tissues. Stem cells are undifferentiated cells that exist in many tissues of embryos and adult mammals. In embryos, blastocyst stem cells are the source of cells which differentiate to form the specialized tissues and organs of the developing fetus. In adults, specialized stem cells in individual tissues (or mulitpotent cells) are the source of new cells which replace cells lost through cell death due to natural attrition, disease or injury. No stem cell is common to all tissues in adults. Rather, the term “stem cell” in adults describes different groups of cells in different tissues and organs with common characteristics.

Until recently, there was little evidence in mammals that multipotent cells such as blood stem cells could change course and produce skin cells, liver cells or any cell other than a blood stem cell or a specific type of blood cell; however, research in animals is leading scientists to question this view. See http://www.nih.gov/news/stemcell/primer.htm.

In animals, it has been shown that some adult stem cells previously thought to be committed to the development of one line of specialized cells are able to develop into other types of specialized cells. For example, recent experiments in mice suggest that when neural stem cells were placed into the bone marrow, they appeared to produce a variety of blood cell types. In addition, studies with rats have indicated that stem cells found in the bone marrow were able to produce liver cells. These exciting findings suggest that even after a stem cell has begun to specialize, the stem cell may, under certain conditions, be more flexible than first thought. For more information, see http://www.nih.gov/news/stemcell/primer.htm.

With the foregoing in mind, the invention herein disclosed provides a novel device useful in oseteoinduction, as well as a method for using the same. These and other advantages of the invention, as well as additional inventive features, will be apparent from the description of the invention provided herein.

### BRIEF SUMMARY OF THE INVENTION

The invention provides a novel type of surgical implant, and processes for producing same, useful upon implantation into a body site for osteoinduction. It is an object of the present invention to provide a novel process for producing multiple centers of cartilage and bone induction through the implantation of artificially perforated organic bone matrix augmented by live human undifferentiated cells suspended in a non-toxic gel. The present inventive process essentially comprises: 1) preparing a non-toxic gel-like suspension of live human undifferentiated cells, 2) forming a plurality of artificial perforations in an organic bone matrix to be surgically implanted, 3) populating the perforations in the organic bone matrix with the live human undifferentiated cells suspended in the non-toxic gel, and 4) implanting the artificially perforated organic bone matrix augmented with live human undifferentiated cells suspended in the non-toxic gel, the augmented bone matrix being accepted by the body, and the implantation of the augmented bone matrix resulting in the transformation of the undifferentiated cells positioned within the perforations into differentiated cartilage and bone cells.

### DETAILED DESCRIPTION OF THE INVENTION

The Artificially Perforated Bone Matrix

U.S. Pat. No. 4,932,973 discloses cartilage and bone induction by artificially perforated organic bone matrix, and discloses the process by which artificially perforated bone matrix is made. That patent in its entirety is hereby incorporated by reference. The process set forth therein is summarized as follows.

Organic bone matrix may be produced using any one of the known prior art methods which may include any one of the following steps. A whole or a part of a bone is harvested from any vertebrate. It can then be conserved by any known conservation method. Partial or complete demineralization of the bone is carried out to cause decalcification by subjecting the bone to treatment with various acids, chelating agents, electrolysis, or any combination of the foregoing. Finally, either prior to or after demineralization of the bone, fixation and different physical and chemical processing is done. The prepared bone or organic bone matrix is then processed to form a plurality of artificial perforations in the form of continuous channels therein, which may be formed by drilling, laser, puncture or the like process. The perforations may be of various shapes, such as, but not limited to, circular, triangular, multiangled, irregular, slit-like or any combination of the foregoing. Preferably, the perforations are in the form of continuous channels, extend from one surface of the matrix to the opposite surface.

The number of perforations in the organic bone matrix may vary; multiple perforations, however, are preferable. Multiple perforations produce a substantial increase in the ability of the organic bone matrix to induce cartilage and bone formation after implantation because each perforation, populated with live human undifferentiated cells, becomes an individual center of cartilage and bone induction.

The perforations in the organic bone matrix need not be uniform in size or shape, nor do they need to be uniformly concentrated over the surface area of the bone matrix.

The Suspension

The present invention improves on the artificially perforated organic bone matrix previously disclosed in U.S. Pat. No. 4,932,973, by augmenting said bone matrix with live, human undifferentiated cells suspended in a non-toxic gel, providing a new implant optimal for use in oseteoinduction.

The undifferentiated cells used in the present invention may be derived from any human sources, including, but not limited to: blood, bone marrow, spleen, adipose tissue, placenta, umbilical cord, or embryo. The cells are suspended in a non-toxic gel, suitable for use in surgical implantation. Any non-toxic gel or putty-like substance may be suitable for purposes of the present invention. Preferably, a bone gel, a gel comprising a suspension of demineralized bone material, is used to suspend the undifferentiated cells. Various bone gels disclosed in the prior art may be used in the present invention, a few of which are hereinafter discussed.

U.S. Pat. No. 5,073,373, hereby incorporated by reference, discloses a simple mixture of glycerol and lyophilized, demineralized bone powder of a particle size in the range of 0.1 cm to 1.2 cm (1000 microns to 12,000 microns), commercially supplied under the trademark GRAFTON®, and suitable in the present invention for purposes of suspending undifferentiated cells.

U.S. Pat. No. 5,290,558, also hereby incorporated by reference, discloses a flowable demineralized bone powder composition using a osteogenic bone powder with a large particle size ranging from about 0.1 to about 1.2 cm, mixed with a low molecular weight polyhydroxy compound possessing from 2 to about 18 carbons including a number of classes of different compounds such as monosaccharides, disaccharides, water dispersible oligosaccharides and polysaccharides. Such bone gel may also be used in the present invention to suspend live human undifferentiated cells.

Another bone gel that may be used in the present invention for suspending undifferentiated cells is disclosed in U.S. Pat. No. 4,172,128, which discloses demineralized bone material mixed with a carrier used to reconstruct tooth or bone material, and made by adding a mucopolysaccharide to a mineralized bone colloidal material. The composition is formed from a demineralized coarsely ground bone material, which may be derived from human bones and teeth, dissolved in a solvent forming a colloidal solution to which is added a physiologically inert polyhydroxy compound such as mucopolysaccharide or polyuronic acid in an amount which causes orientation when hydrogen ions or polyvalent metal ions are added to form a gel. The gel will be flowable at elevated temperatures above 35° C. and will solidify when brought down to body temperature.

Another prior art product that may be used in the present invention for suspending undifferentiated cells is a formulation comprising demineralized allograft bone particles suspended in collagen.

Yet another prior art bone gel that may be used in the present invention for suspending undifferentiated cells is disclosed in U.S. Pat. No. 6,030,635, which describes a method for making a bone putty or gel in which bone powder is suspended in a carrier that is selected from the group consisting of sodium hyaluronate, chitosan and N,O-carboxymethylchitosan, in water solution. The use of other hydrogels is also disclosed.

In a preferred embodiment of the present invention, the gel used to suspend the live human undifferentiated cells is a bone gel prepared according to the following method. A supernatant may be formed by dissolving demineralized allograft bone matrix, in the form of cubes, shavings, or powders, in either water or a solution comprising water and at least one component normally found in animal blood serum (e.g., sodium chloride, potassium phosphate, sodium bicarbonate, or the like) at a temperature of above about 25° C. Preferably, dissolution is enhanced by the use of agitation and/or ultrasound. The dissolution may also be enhanced by use of pressure. Preferably the pressure will be at least about 15 psi, for a period of time. The resulting viscous supernatant is then cooled. Most preferably the supernatant is obtained by heating to a temperature between about 85° C. and about 100° C. As the demineralized bone dissolves in the water, a viscous supernatant is formed from the demineralized bone. The bone dissolution is continued under the elevated temperature for a period of time sufficient to generate a supernatant having a viscosity above about 1 centipoise; more particularly, the heating is continued to the point where a thick, viscous liquid has been formed, which usually takes place between about 24 and about 120 hours. The supernatant is then allowed to cool. If the heated solution is subjected to an elevated pressure of at least about 15 psi, more preferably between about 15 psi and about 90 psi, the bone is dissolved more readily. The use of pressure to dissolve the demineralized bone matrix is faster, in that the process, depending on the amount of pressure used, usually only needs to be carried out from between about 1 hour to about 8 hours. The further advantage lies in that there may not need to be any mechanical agitation, as the elevated pressure compensates for this. Usually the amount of dissolved demineralized bone will be from about 0.5 to about 25 percent, by weight, based on the total weight of water and bone, preferably from about 5 to about 10 percent. The demineralized bone used to make the supernatant may be in any physical form, such as chips, shavings or particles. The form of the demineralized bone used to make the supernatant is not critical, as it ultimately will be dissolved. The use of smaller particles will aid in the dissolution process. Sufficient demineralized bone is dissolved to increase the viscosity of the water. Preferably the viscosity is increased to from about 2 to about 100 centipoises, preferably from about 40 to about 200 centipoises. Alternatively sufficient demineralized bone is dissolved to convert the carrier into a gel or gel-like structure. The water may be sterile water for injection or sterile saline solution or may comprise other components, such as those normally found in blood, such as BSS balanced salt solution, containing 140 mM NaCl, 5.4 mM KCl, at a pH of 7.6. Soluble calcium can be attracted to the surgical site by using a sodium phosphate buffer of pH 7.2 in lieu of the isotonic saline. The phosphate buffer will attract calcium cations to the site from the surrounding healthy bone and create an equilibrium concentration of the calcium precisely at the site of healing where it is most desirable to grow new bone. Once the viscous, near gel-like supernatant is produced and cooled to near room temperature, the live human undifferentiated cells are mixed in, suspending the cells within the bone gel.

The Augmented Bone Matrix

Once a suitable gel suspension of live human undifferentiated cells is produced, the artificially perforated bone matrix is then contacted with the suspension in order to populate perforations on the bone matrix with the undifferentiated cells. Any suitable means of populating perforations on a bone matrix may be used, including, but not limited to: 1) manual application, 2) manual application with the aid of a spatula, 3) submerging the bone matrix within the suspension, 4) centrifuging the bone matrix submerged within a suspension, or 5) any other suitable method in the art.

Once the perforations on a bone matrix are sufficiently populated with the live human undifferentiated cells suspended in gel, the augmented artificially perforated bone matrix may be implanted within a patient's body, resulting in eventual transformation of the undifferentiated cells positioned within the perforations into differentiated cartilage and bone cells.

The following examples further illustrate the invention but, of course, should not be construed as in any way limiting its scope.

This example demonstrates a method making the present invention.

### EXAMPLE I

Demineralized bone powder with particle size of 45-125 μm is placed in distilled water boiling at 100° C. The solution is constantly agitated by magnetic stirring and/or ultrasound for 72 hours, at which point a viscous, near-gel like supernatant has been produced. The supernatant is then set out for several hours to cool, and then mixed with live undifferentiated cells obtained from human bone marrow.

Next, a perforated organic bone matrix was prepared in accordance with the invention taught herein and as disclosed in U.S. Pat. No. 4,932,973. The perforated organic bone matrix was then submerged in the bone gel suspension of cells and centrifuged at low speed, resulting the in the perforations on the bone matrix becoming populated with live undifferentiated cells. An augmented bone matrix was thus obtained, optimal for use in osteoinduction upon surgical implantation.

### EXAMPLE II

This example demonstrates a method making the present invention.

Demineralized bone powder with a particle size of 45-125 μm is placed in distilled boiling water and autoclaved at a temperature of 110-115° C. and at a pressure of 20-22 psi for about 3 hours, at which point a viscous supernatant is produced. The supernatant is then set out for several hours to cool, and then mixed with live undifferentiated cells obtained from human bone marrow.

Next, a perforated organic bone matrix was prepared in accordance with the invention taught herein and as disclosed in U.S. Pat. No. 4,932,973. The perforated organic bone matrix was then submerged in the bone gel suspension of cells and centrifuged at low speed, resulting the in the perforations on the bone matrix becoming populated with live undifferentiated cells. An augmented bone matrix was thus obtained, optimal for use in osteoinduction upon surgical implantation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An implant for inducing the formation of bone and cartilage within a body, said implant consisting essentially of organic bone matrix having discrete predetermined perforations populated with live human undifferentiated cells suspended in a gel; said populated perforations, after implantation within said body, being capable of stimulating the transformation of said cells into differentiated cartilage and bone cells.

2. The implant of claim 1, wherein said gel is a bone gel comprising demineralized bone matrix and water.

3. The implant of claim 1, wherein said gel is a bone gel comprising demineralized bone matrix and human blood serum or any component thereof.

4. The implant of claim 1, wherein said live undifferentiated cells are derived from blood.

5. The implant of claim 1, wherein said live undifferentiated cells are derived from bone marrow.

6. The implant of claim 1, wherein said live undifferentiated cells are derived from a spleen.

7. The implant of claim 1, wherein said live undifferentiated cells are derived from adipose tissue.

8. The implant of claim 1, wherein said live undifferentiated cells are derived from a placenta.

9. The implant of claim 1, wherein said live undifferentiated cells are derived from an umbilical cord.

10. The implant of claim 1, wherein said live undifferentiated cells are derived from an embryo.

11. A method of preparing the implant of claim 1, said method comprising:
(a) preparing a gel suitable for suspending live human undifferentiated cells, said gel being non-toxic to humans upon implantation;
(b) mixing live human undifferentiated cells with said gel to prepare a suspension of said cells;
(c) preparing an organic bone matrix having discrete predetermined perforations; and
(d) causing said perforations within said organic bone matrix to be populated with said live human undifferentiated cells suspended in said gel.

12. The method of claim 11, wherein the gel is a bone gel prepared by:
(a) contacting demineralized bone matrix with water at an elevated temperature sufficient to aid in the dissolution of the bone matrix, and optionally at an elevated pressure to aid in the dissolution of the bone matrix, for a period of time sufficient to provide a supernatant having a near gel-like viscosity; and
(b) cooling the resulting supernatant to about room temperature.

13. The method of claim 12, wherein said demineralized bone matrix is in the form of a bone powder having an average particle size of about 10 microns to about 850 microns.

14. The method of claim 12, wherein said demineralized bone matrix is in the form of a bone powder having an average particle size of about 45 microns to about 125 microns.

15. The method of claim 12, wherein said demineralized bone matrix is contacted:
(i) with water at a temperature above about 25 degrees Centigrade with agitation; or
(ii) with water at a temperature above about 25 degrees Centigrade and at a pressure above about 15 psi.

16. The method of claim 12, wherein said demineralized bone matrix is contacted with water at an average temperature between about 85 and about 100 degrees Centigrade, for a period of time between about 24 and about 120 hours.

17. The method of claim 15, wherein the demineralized bone matrix is contacted with water at an average temperature between about 105 and about 200 degrees Centigrade, and at a pressure between about 15 to about 90 psi, for about 1 to about 8 hours.

18. The method of claim 17, wherein:
(a) the demineralized bone matrix is in the form of a bone powder with an average particle size between 45-124 microns; and
(b) the demineralized bone matrix is contacted with water at an average temperature between about 110-115 degrees Centigrade, and at a average pressure between 20 psi and 22 psi, for about 3 hours.

19. The method of claim 11, wherein the gel is a bone gel prepared by:
(a) dissolving demineralized bone matrix in:
(i) a solution comprising water and human blood serum or any component thereof at a temperature of above about 25 degrees Centigrade with agitation; or
(ii) a solution comprising water and human blood serum or any component thereof at a temperature of at least about 25 degrees Centigrade and a pressure of at least about 15 psi;
for a period of time sufficient to provide a supernatant having a near gel-like viscosity; and
(b) cooling the resulting supernatant to about room temperature.

20. The method of claim 19, wherein said demineralized bone matrix is in the form of a bone powder having an average particle size of about 10 microns to about 850 microns.

21. The method of claim 19, wherein said demineralized bone matrix is in the form of a bone powder having an average particle size of about 45 microns to about 125 microns.

22. The method of claim 19, wherein said demineralized bone matrix is contacted with said solution at an average temperature between about 85 and about 100 degrees Centigrade, for a period of time between about 24 and about 120 hours.

23. The method of claim 19, wherein said demineralized bone matrix is contacted with said solution at an average temperature between about 105 and about 200 degrees Centigrade, and at a pressure between about 15 to about 90 psi, for about 1 to about 8 hours.

24. The method of claim 11, wherein said live undifferentiated cells are derived from blood.

25. The method of claim 11, wherein said live undifferentiated cells are derived from bone marrow.

26. The method of claim 11, wherein said live undifferentiated cells are derived from a spleen.

27. The method of claim 11, wherein said live undifferentiated cells are derived from adipose tissue.

28. The method of claim 11, wherein said live undifferentiated cells are derived from a placenta.

29. The method of claim 11, wherein said live undifferentiated cells are derived from an umbilical cord.

30. The method of claim 11, wherein said live undifferentiated cells are derived from an embryo.

31. The method of claim 11, wherein step (d) is achieved by:
a. submerging said organic bone matrix having discrete predetermined perforations into said suspension of live human undifferentiated cells, and centrifuging said submerged organic bone matrix for a period of time sufficient to cause said perforations within said organic bone matrix to be populated with said live human undifferentiated cells suspended in said gel.

32. A method for inducing the formation of bone and cartilage within a body comprising implanting within said body a matrix material comprising organic bone matrix having discrete predetermined perforations populated with live human undifferentiated cells suspended in a non-toxic gel, said matrix, after implantation within said body, being capable of stimulating the transformation of said cells into differentiated cartilage and bone cells.
